(19) United States
(12) Patent Application Publication
(10) Pub. No.: US 2004/0143313 A1
(43) Pub. Date: Jul. 22, 2004

(54) Joint protector

(76) Inventors: Hsiu Yu Chang, Taichuang (TW); Li Ying Hung, Nantou Hsien (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/347,198

(22) Filed: Jan. 21, 2003

(51) Int. Cl. A61N 1/04

(52) U.S. Cl. 607/108; 607/148; 607/149; 607/152

(57) ABSTRACT

A joint protector includes a band surrounding a human body and having an inner side and an outer side. A power supplier is securely attached to the outer side of the band. A flexible heating pad is attached to the inner side of the band and electrically connected to the power supplier. The flexible heating pad provides heat energy to warm the user's skin and reduce pain due to the electric resistance of the flexible heating pad. An inner layer is securely adhered on the inner side of the band for covering the flexible heating pad.

### BACKGROUND OF THE INVENTION

1. Field of the Invention

The present invention relates to a protector, and more particularly to joint protector.

2. Description of Related Art

A conventional joint protector in accordance with the prior art comprises band adapted for being mounted around the joint of human body to prevent the joint from a sport injure. A hot compress further is provided to the player before sports to prevent the joint from a sport injure and after sports to eliminate player from tiredness. However, a hot source, such as a hot towel or a chemical warmer, is usually used to apply on user's skin when taking a hot compress such that the user cannot move arbitrarily to prevent the hot towel or the chemical warmer from detaching from the user's skin.

Consequently, the joint protector is altered that is substantially the combination of the band and a heater for easy use. With reference to FIG. 5, the conventional heater (50) has a heating pad (51) fixed in the joint protector and made of nonmetal material. The heating pad (51) has two opposite sides each having a conduct wire (52) partially securely received in the heating pad (51). Each conduct wire (52) has a free end electrically connected to a power source. Consequently, the heating pad (51) can cause heat energy to warm the user's skin and reduce pain due to the electric resistance of the heating pad (51).

The conventional heater (50) of joint protector has only two electrodes (conduct wire) received in the heating pad (51) such that the heating pad (51) should be shut down even burnout when anyone of the two electrodes has a broken circuit.

The present invention has arisen to mitigate and/or obviate the disadvantages of the conventional joint protector.

### SUMMARY OF THE INVENTION

The main objective of the present invention is to provide an improved joint protector.

To achieve the objective, the joint protector in accordance with the present invention includes a band surrounding a human body and having an inner side and an outer side. A power supplier is securely attached to the outer side of the band. A flexible heating pad is attached to the inner side of the band and electrically connected to the power supplier. The flexible heating pad provides heat energy to warm the user's skin and reduce pain due to the electric resistance of the flexible heating pad. An inner layer is securely adhered on the inner side of the band for covering the flexible heating pad.

Further benefits and advantages of the present invention will become apparent after a careful reading of the detailed description with appropriate reference to the accompanying drawings.

### BRIEF DESCRIPTION OF THE DRAWINGS

FIG. 1 is a perspective view of a joint protector in accordance with the present invention;

FIG. 2 is a perspective view of another embodiment of a joint protector in accordance with the present invention;

FIG. 3 is a plan view of a heating pad of the joint protector in accordance with the resent invention;

FIG. 4 is a cross-section view of the heat pad in FIG. 3 along line 4-4; and

FIG. 5 is perspective view of a conventional heating pad of a joint protector in accordance with the prior art.

### DETAILED DESCRIPTION OF THE INVENTION

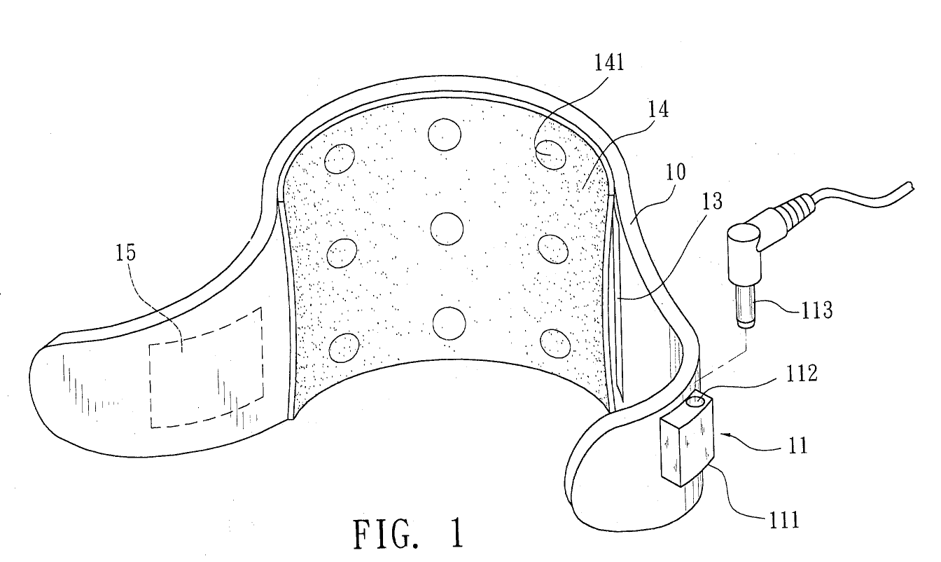

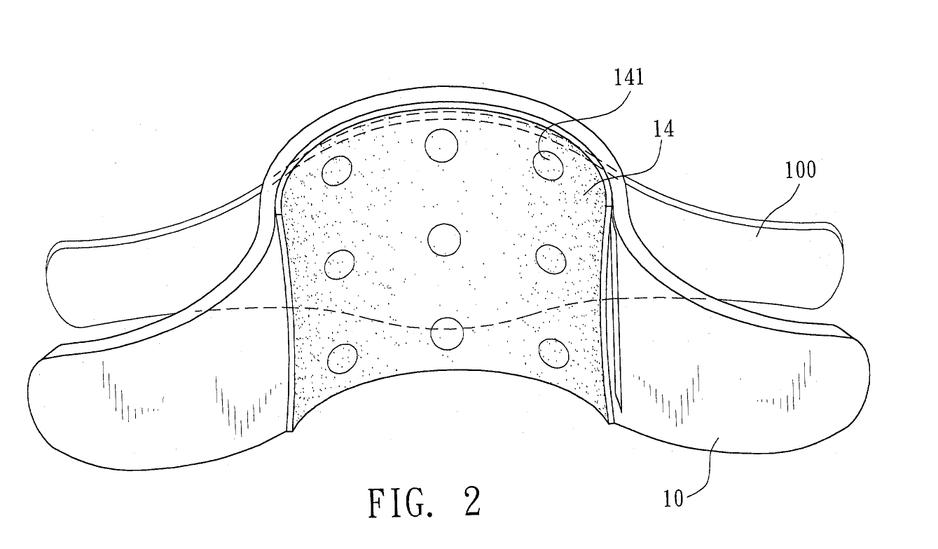

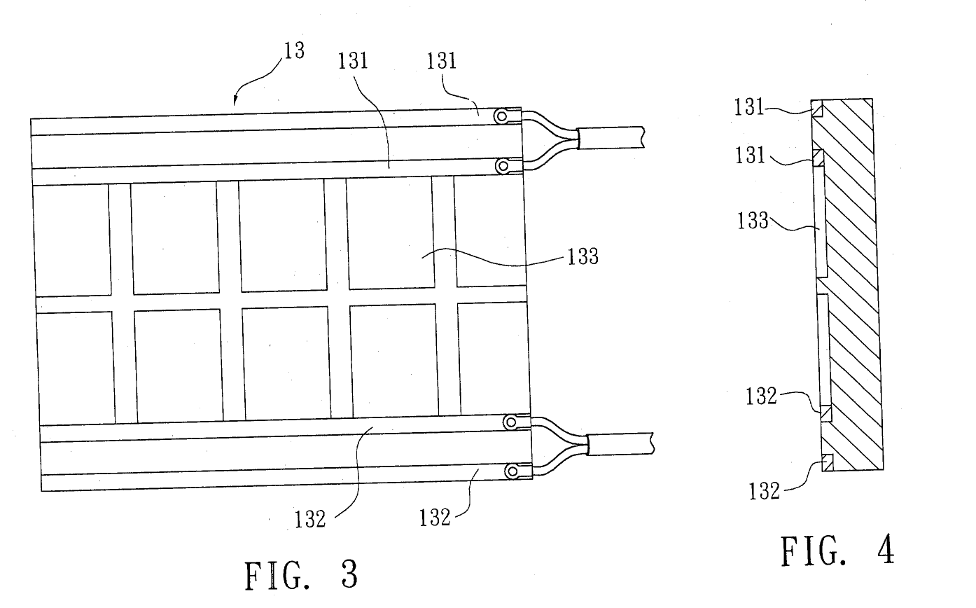

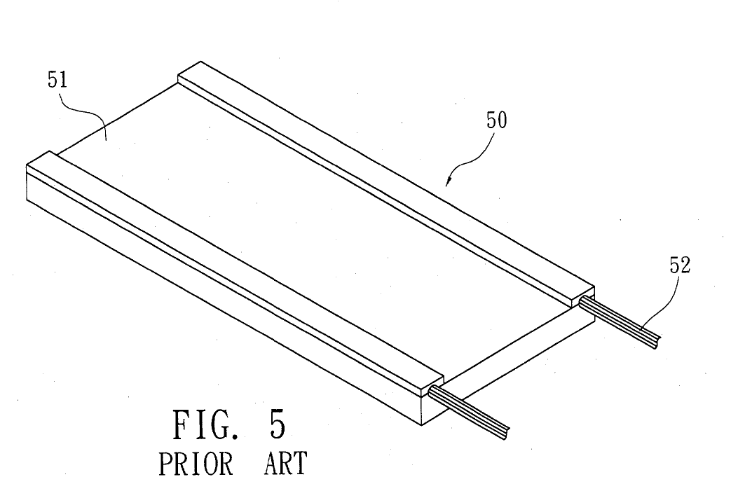

Referring to the drawings and initially to FIGS. 1-3, a joint protector in accordance with the present invention comprises band (10) adapted to surrounding a human body and having an inner side and an outer side. An extra band (100) is attached to the outer side of the band (10) to promote the fixing effect of the present invention. A power supplier (11) is securely attached to the outer side of the band (10). The power supplier (11) has a casing (111) attached to the outer side of the band (10) for receiving battery (not shown) when the joint protector is used outdoors and a socket (112) defined in the casino (111) to receive a plug (113) that is electrically connected to a power source when the joint protector is used indoors or the battery in the casing (111) is exhausted. A flexible heating pad (12) is attached to the inner side of the band (10) and electrically connected to the power supplier (11). The heating pad (12) can cause heat energy to warm the user's skin and reduce pain due to the electric resistance of the heating pad (12). Further with reference to FIG. 4, the heating pad (13) has two first electrodes (131) securely attached to a surface of the heating pad (13) along a first side of the heating pad (13) and two second electrodes (132) securely attached to the surface of the heating pad (13) along a second side of the heating pad (13). The first side and the second side of the heating pad (13) are parallel and opposite each other. Each electrode (131, 132) is parallel to one another and electrically connected to the power supplier (11). Multiple recesses (133) are defined in the surface of the heating pad (13) and between the first electrodes (131) and the second electrodes (132). Consequently, the joint protector of the present invention can result a convecting air current between the heating pad (13) and the band (10) such that the user will feel warmer and saving energy. An inner layer (14) is securely adhered on the inner side of the band (10) for covering the heating pad (13). The inner layer (14) has multiple exciters (141) secured on the inner layer (14) and electrically connected to the power supplier (11). The exciter (141) is provided to touch the user's skin for shooting an electric current that can excite the points in the human body to achieve an acupuncture effect for reducing pain caused by a sports injure. A vibrator (15) is secured on the outer side of the band (10) and electrically connected to the power supplier (11) such that the joint protector can provide a massage effect to the user when the vibrator is operated.

Although the invention has been explained in relation to its preferred embodiment, it is to be understood that many other possible modifications and variations can be made without departing from the spirit and scope of the invention as hereinafter claimed.

What is claimed is:

1. A joint protector comprising:
a band adapted to surround a human body and having an inner side and an outer side;
a power supplier securely attached to the outer side of the band;
a flexible heating pad attached to the inner side of the band and electrically connected to the power supplier, the flexible heating pad adapted to provide heat energy to warn the user's skin and reduce pain due to the electric resistance of the flexible heating pad, the flexible heating pad includes:
two first electrodes securely attached to a surface of the flexible heating pad and along a first side of the flexible heating pad, each first electrode electrically connected to the power supplier; and
two second electrodes securely attached to the surface of the flexible heating pad and along a second side of the flexible heating pad, each second electrode electrically connected to the power supplier; and
an inner layer securely adhered on the inner side of the band for covering the flexible heating pad.

2. The joint protector as claimed in claim 1, wherein the flexible heating pad comprises multiple recesses defined in the surface thereof and between the first electrodes and the second electrodes such that a convecting air current is formed between the flexible heating pad and the band.

3. The joint protector as claimed in claim 1, wherein the inner layer comprises multiple exciters secured thereon and electrically connected to the power supplier, the exciters adapted to touch the user's skin for shooting an electric current to achieve an acupuncture effect.

4. The joint protector as claimed in claim 2, wherein the inner layer comprises multiple exciters secured thereon and electrically connected to the power supplier, the exciters adapted to touch the user's skin for shooting an electric current to achieve an acupuncture effect.

5. The joint protector as claimed in claim 1 further comprising a vibrator secured on the outer side of the band and electrically connected to the power supplier such that the joint protector can provide a massage effect when the vibrator is operated.

6. The joint protector as claimed in claim 2 further comprising a vibrator secured on the outer side of the band and electrically connected to the power supplier such that the joint protector can provide a massage effect when the vibrator is operated.

7. The joint protector as claimed in claim 3 further comprising a vibrator secured on the outer side of the band and electrically connected to the power supplier such that the joint protector can provide a massage effect when the vibrator is operated.

8. The joint protector as claimed in claim 4 further comprising a vibrator secured on the outer side of the band and electrically connected to the power supplier such that the joint protector can provide a massage effect when the vibrator is operated.

9. The joint protector as claimed in claim 1, wherein the power supplier comprises a casing secured on the outer side of the band and adapted to receive battery when the joint protector is used outdoors, and a socket defined in the casing, the socket adapted to receive a plug that is electrically connected to a power source when the joint protector is used indoors.

10. The joint protector as claimed in claim 2, wherein the power supplier comprises a casino secured on the outer side of the band and adapted to receive battery when the joint protector is used outdoors, and a socket defined in the casing, the socket adapted to receive a plug that is electrically connected to a power source when the joint protector is used indoors.

11. The joint protector as claimed in claim 3, wherein the power supplier comprises a casing secured on the outer side of the band and adapted to receive battery when the joint protector is used outdoors, and a socket defined in the casing, the socket adapted to receive a plug that is electrically connected to a power source when the joint protector is used indoors.

12. The joint protector as claimed in claim 4, wherein the power supplier comprises a casing secured on the outer side of the band and adapted to receive battery when the joint protector is used outdoors, and a socket defined in the casing, the socket adapted to receive a plug that is electrically connected to a power source when the joint protector is used indoors.

13. The joint protector as claimed in claim 5, wherein the power supplier comprises a casing secured on the outer side of the band and adapted to receive battery when the joint protector is used outdoors, and a socket defined in the casing, the socket adapted to receive a plug that is electrically connected to a power source when the joint protector is used indoors.

14. The joint protector as claimed in claim 6, wherein the power supplier comprises a casing secured on the outer side of the band and adapted to receive battery when the joint protector is used outdoors, and a socket defined in the casing, the socket adapted to receive a plug that is electrically connected to a power source when the joint protector is used indoors.

15. The joint protector as claimed in claim 7, wherein the power supplier comprises a casing secured on the outer side of the band and adapted to receive battery when the joint protector is used outdoors, and a socket defined in the casing, the socket adapted to receive a plug that is electrically connected to a power source when the joint protector is used indoors.

16. The joint protector as claimed in claim 8, wherein the power supplier comprises a casing secured on the outer side of the band and adapted to receive battery when the joint protector is used outdoors, and a socket defined in the casing, the socket adapted to receive a plug that is electrically connected to a power source when the joint protector is used indoors.
